(19) United States
(12) Patent Application Publication
(10) Pub. No.: US 2015/0105426 A1
(43) Pub. Date: Apr. 16, 2015

(54) TOPICAL EMULSIONS BASED ON MIXTURES OF LOCAL EUTECTIC ANAESTHETICS AND FATTY ACIDS AS ANALGESICS, ANTALGICS, OR AS SEXUAL RETARDANTS

(71) Applicant: UNIVERSITE PARIS DESCARTES, Paris (FR)

(72) Inventors: Mathieu Lazerges, Paris (FR); Philippe Espeau, Boissy-Saint-Leger (FR); Sylvie Sophie Crauste-Manciet, Paris (FR); Denis Brossard, Antony (FR); Yohann Corvis, Suresnes (FR); Florence Agnely, Bourg-La-Reine (FR); Nicolas Huang, Paris (FR)

(73) Assignee: UNIVERSITE PARIS DESCARTES, Paris (FR)

(21) Appl. No.: 14/363,568

(22) PCT Filed: Dec. 4, 2012
(86) PCT No.: PCT/FR2012/052799

§ 371 Date: Nov. 4, 2014

(30) Foreign Application Priority Data
Dec. 7, 2011 (FR) 11/61264

(51) Int. Cl. A61K 9/107; A61K 31/167; A61K 9/00; A61K 47/12; A61K 47/32; A61K 47/24; A61K 47/38; A61K 47/10; A61K 47/26; A61K 47/36; A61K 31/445; A61K 47/14

(52) U.S. Cl. CPC A61K 9/107; A61K 31/445; A61K 31/167; A61K 9/0014; A61K 47/12; A61K 47/14; A61K 47/24; A61K 47/38; A61K 47/10; A61K 47/26; A61K 47/36; A61K 47/32
USPC 514/330; 514/626

(57) ABSTRACT

The invention relates to novel formulations for topical local anaesthesia and for the use thereof as analgesics, antalgics, or as sexual retardants.

The invention relates to novel formulations for topical local anaesthesia, pain alleviation or as sexual retardant.

Anesthetizing creams are applied to the skin before taking a blood sample, an injection or in dermatology before conducting surgical or laser procedure. Two medicinal products currently available in France are EMLA® and its generic Anesderm®.

The oil phase of the EMLA® cream used at the present time for topical local anaesthesia is a liquid eutectic mixture of anaesthetics (EMLA®: Eutectic Mixture of Liquid Anaesthetic). EMLA® also has an antalgic effect (i.e. attenuation of pain) (Archives de Pédiatrie, 2004, 11, 921-925).

EMLA is a mixture containing equal weights of two local anaesthetics: prilocaine (CAS 721-50-6) and lidocaine (CAS 137-58-6). EMLA is liquid at ambient temperature whereas the two pure compounds are solid. The mutual lowering of the melting point of the two compounds in the mixture results from a physical effect which does not modify the chemical nature of the constituents of the mixture: the eutectic effect. This liquid phase is both the active ingredient and the oil phase of an oil-in-water emulsion whose pharmaceutical formulation is a cream: EMLA® cream. The oil phase of EMLA® cream diffuses through the Stratum Corneum which is the main physiological barrier of human skin.

EMLA® has an adverse effect however caused by prilocaine: it may cause methaemoglobinaemia in particular in infants and young children. This is caused by conversion of haemoglobin to methaemoglobin at toxic thresholds, induced by metabolites of prilocaine (Best Practice & Research Clinical Anaesthesiology, 2003, 17, 111-136, Cox et al.). Several complications induced by prilocaine have been reported (Eur. J. Pediatr., 1999, 158, 785-788, Frey at al.; The Journal of Emergency Medicine, 2004, 26, 85-88, Hahn et al.).

To overcome this problem, a liposomal formulation has been developed and is marketed in the United States: ELA-Max®, but it is not marketed in France. Double-blind clinical trials on the anaesthetizing effect of EMLA® and ELA-Max® on the skin before intravenous injection have shown that there are no significant differences between the two formulations (Pediatric Anesthesia, 2004, 14, 977-982, Koh at al.). Another clinical trial on the anaesthetizing effect on heat-induced pain shows that EMLA® performs better than ELA-Max® (Journal of Dermatologic Treatment 2004, 15, 84-87, Tang at al.).

One of the objectives of the invention is the use of an oil phase with eutectic invariant (PHE) to prepare oil-in-water emulsions.

A second objective of the invention is to provide an oil phase with eutectic invariant (PHE) that is liquid, homogeneous, has high anaesthetic content and does not necessarily contain prilocaine. This oil phase with eutectic invariant (PHE) has advantageous physicochemical properties compared with EMLA in terms of thermodynamic stability and of interest in terms of diffusion through the human Stratum Corneum.

A third objective of the invention is to provide an oil-in-water (O/W) emulsion comprising the said oil phase with eutectic invariant (PHE).

A further objective is to use the said emulsion as local anaesthetic, antalgic or sexual retardant.

The present invention concerns the use of an oil phase with eutectic invariant (PHI) comprising at least one local anaesthetic in a proportion of 10% to 60% by weight relative to the total weight of the oil phase (PHI) and at least one fatty acid in a proportion of 40% to 90% by weight relative to the total weight of the oil phase (PHI) to form an oil-in-water emulsion (O/W) wherein the pH of the hydrophilic phase is particularly between 6 and B, more particularly between 6.5 and 7.5, the said oil phase (PHI) being of weak ionic type and in homogenous liquid form stable at a temperature of between 0° C. and 37° C., provided that when the said local anaesthetic is mepivacaine or bupivacaine either the said local anaesthetic is associated with at least one second local anaesthetic different thereto, or at least two different fatty acids are used to obtain a eutectic invariant.

An [symbol] oil-in-water emulsion [symbol] (O/W) is formed of two liquid phases: a discontinuous lipophilic phase, in particular an oil, dispersed in droplet form in a continuous hydrophilic phase.

A [symbol] local anaesthetic [symbol] is an active ingredient which reversibly inhibits the propagation of signals along nerves by blocking sodium channels and which produces anaesthetizing effects on part of a patient's body without the need for a general anaesthetic.

A [symbol] fatty acid [symbol] is a hydrophobic compound with C₅ to C₂₄ carboxylic acid function, preferably C₁₂ to C₂₄ with aliphatic, straight-chain or branched, saturated or unsaturated chain.

Preferably the fatty acid is scarcely soluble in the aqueous phase and does not form co-crystals with the local anaesthetic.

The expression [symbol] fatty acid [symbol] may also designate a fatty acid analogue of similar hydrophobic-hydrophilic balance formed, like the fatty acid, of a hydrophobic hydrocarbon part and of a carboxylic acid group but additionally including another chemical function such as an ester function or alcohol function for example.

By the expression [symbol] oil phase with eutectic invariant [symbol] it is meant that at a constant temperature, known as eutectic, the total melting of at least one of the two solid constituents of the mixture is observed, irrespective of the proportion of the compounds contained in the mixture. By solid is meant either one of the two pure bodies if there is total immiscibility between them in the solid state, or a solid solution formed by a homogeneous mixture of the pure constituents. The particular composition in which the melting of all the solid constituents of the mixture is total at eutectic temperature is called a eutectic composition. An essential property of mixtures with eutectic invariant, for solids non-miscible in the solid state, is the lowering of the melting point of the pure compounds constituting the mixture, in other words the liquidus curve, over the entire composition range. Either side of the eutectic point, and above eutectic temperature, only one of the two pure constituents is in equilibrium with a homogeneous liquid. This lowering of temperature is maximal for a eutectic composition. If there is partial miscibility in the solid state, the liquidus curve then corresponds to an equilibrium between a homogeneous liquid and a solid solution whose composition varies with temperature. It nevertheless remains true that the temperature of this liquidus, at which the entirety of the said solid solution disappears, is always lower than the melting point of the pure body from which the corresponding liquidus curve originates.

The oil phases of the invention are therefore in liquid form irrespective of the anaesthetics and fatty acids used.

If the obtaining of a liquid oil phase is not possible between an anaesthetic and a fatty acid, the one skilled in the art must then add another anaesthetic and/or another fatty acid to obtain a liquid oil phase.

The local anaesthetics of the invention have a strong amine function and this is almost entirely in the form of a non-salified base, and the fatty acid contained in the oil phase is in almost entirely non-salified acid form.

The expression [symbol] being of weak ionic nature [symbol] means that the amine function of lidocaine is not in its acid form RR′NH⁺ and that the carboxylic acid function of lidocaine is not in its basic form R″COO⁻. The salt content is negligible, less than a value of 10% by weight, in particular less than a value of 5% by weight and more particularly less than a value of 1% by weight.

The term [symbol] homogenous [symbol] according to the invention means that the entirety or the anaesthetics (s) and the entirety of the fatty acid(s) present in the liquid mixture are miscible.

The term [symbol] stable [symbol] means that the oil phase remains liquid and homogeneous, without it chemical nature being deteriorated, ideally indefinitely. In practice, the eutectic lidocaine-lauric acid mixture in a closed bottle stored away from light remains in its transparent liquid form for at least 10 months, in particular at least 18 months.

The inventors have determined that some fatty acids of defined molar mass can advantageously be associated with lidocaine to formulate liquid phases with eutectic invariant (PHE) that are homogeneous and with high lidocaine content, and (O/W) emulsions including these oil phases (PHE).

One advantage is the choice of local anaesthetic, which in the event of exclusion of prilocaine, allows methaemoglobinaemia to be prevented in premature babies, infants and young children.

The idea of associating a fatty acid with lidocaine to formulate an oil phase with eutectic invariant (PHE) is original and judicious. The fatty acids include a carboxylic acid function (COOH) which, via specific hydrogen double bonds, is known to associate with primary or secondary amide functions (NHCO). It happens that the local anaesthetics in the amide ester class such as lidocaine include said function. But it also happens that in numerous cases these specific hydrogen associations lead to the formation of solid co-crystals between the two compounds. For the formulation of the oil phase of an emulsion the spontaneous formation of solid co-crystals would be adverse. Nonetheless fatty acids have another asset: they include a hydrocarbon chain whose molecular symmetry, whether straight-chain or branched, is highly remote from that of local anaesthetics e.g. lidocaine, which includes a conjugated system and is more compact. Said difference in molecular symmetry strongly reduces the probability of co-crystal formation. However, these specific hydrogen interactions remain in the liquid state in fine, the effect of which is to significantly lower the eutectic melting temperature. The advantage of these mixtures, directly following the foregoing, is that they therefore have a low eutectic melting temperature, lower than that of all the binary eutectic mixtures of lidocaine reported to date, which broadens the storage temperature range and range of use of these formulations: for the invention, the melting temperatures of the eutectic lidocaine-lauric acid and lidocaine-tridecanoic acid mixtures are 5° C. and 6° C. respectively. For the binary mixtures of lidocaine presently known the eutectic melting temperatures are 29° C. and 38° C. for the lidocaine-menthol mixture (Journal of Physical Chemistry B, 2010, 114, 5420-5426, Corvis at al.) and 18° C. for lidocaine-salol mixtures (Thermochimica Acta, 2010, 497, 124-128, Lazerges at al.) and lidocaine-prilocaine (Journal of Pharmaceutical Sciences, 1984, 73, 481-484, Brodin et al.).

Over and above 60% by weight of local anaesthetic it is no longer possible to obtain a liquid oil phase.

Below 10% the proportion of anaesthetic is too small to have any action.

In one advantageous embodiment, the present invention concerns the use of an oil phase such as defined above wherein the local anaesthetic is in a proportion of 10% to 50% by weight of the total weight of the oil phase, and the fatty acid is in a proportion of 50% to 90% by weight relative to the total weight of the oil phase (PHE).

In one advantageous embodiment, the present invention concerns the use of an oil phase such as defined above, wherein the local anaesthetic is in a proportion of 20% to 60% by weight of the total weight of the oil phase, and the fatty acid is in a proportion of 40% to 80% by weight relative to the total weight of the oil phase (PHE).

In one advantageous embodiment, the present invention concerns the use of an oil phase such as defined above wherein the local anaesthetic is in a proportion of 30% to 60% by weight relative to the total weight of the on phase, and the fatty acid is in a proportion of 40% to 70% by weight relative to the total weight of the oil phase (PHE).

In one advantageous embodiment, the present invention concerns the use of an oil phase such as defined above, wherein the local anaesthetic is in a proportion of 30% to 50% by weight relative to the total weight of the oil phase, and the fatty acid is in a proportion of 50% to 70% by weight relative to the total weight of the oil phase (PHE).

In one advantageous embodiment, the present invention concerns the use of an oil phase such as defined above wherein the said local anaesthetic is in a proportion of 40% to 50% by weight, and the said fatty acid in a proportion of 50% to 60% by weight relative to the total weight of the oil phase (PHE), the said oil phase with eutectic invariant being in the form of a homogeneous liquid stable at a temperature of between 0° C. and 37° C.

Preferably, the oily aqueous phase such as defined above comprises at least one local anaesthetic in a proportion of 40%, 41%, 42%, 43%, 44%, 45%, 46%, 47%, 48%, 49% or 50% by weight, and at least one fatty acid in a proportion of 50%, 51%, 52%, 53%, 54%, 55%, 56%, 57%, 58%, 59% or 60% by weight relative to the total weight of the oil phase (PHE).

In one advantageous embodiment, the present invention concerns the use of an oil phase with eutectic invariant (PHE) such as defined above wherein the local anaesthetic is selected from among lidocaine, levobupicaine, prilocaine, articaine, ropivacaine, dibucaine and the mixtures thereof

In premature babies and children aged 0 to 3 years, the said oil phase cannot contain prilocaine on account of risks of methaemoglobinaemia.

The registration numbers of the different local anaesthetics in the [symbol] Chemical Abstract Services [symbol] database (CAS) are the following: lidocaine (137-58-6); mepivacaine (96-88-8); bupivacaine (38396-39-3); levobupicaine (27-262-47-1); prilocaine (721-50-6); articaine (23944-58-1); ripovacaine (84057-95-4); dibucaine (85-79-0).

In one advantageous embodiment the present invention concerns the use of an oil phase with eutectic invariant (PHE) such as defined above wherein the local anaesthetic is lidocaine.

In one advantageous embodiment, the present invention concerns the use of an oil phase with eutectic invariant (PHE) such as defined above wherein the local anaesthetic is lidocaine in a proportion of 50% by weight relative to the total weight of the oil phase (PHE).

In one advantageous embodiment, the present invention concerns the use of an oil phase with eutectic invariant (PHE) such as defined above wherein the local anaesthetic is lidocaine in a proportion of 50% by weight and the fatty acid is in a proportion of 50% by weight relative to the total weight of the oil phase (PHE).

In one advantageous embodiment, the present invention concerns the use of an oil phase with eutectic invariant (PHE) such as defined above wherein the said fatty acid is a saturated or unsaturated, straight-chain or branched fatty acid having 12 to 24 carbon atoms.

The straight-chain, saturated fatty acids having 12 to 24 carbon atoms are the acids of the following general formulas: H₃C—(CH₂)ₙ—COOH wherein n varies from 10 to 22. Examples of straight-chain saturated fatty acids having 12 to 24 carbon atoms, these being non-limiting, are the following: lauric acid or dodecanoic acid (C12:0), tridecylic acid or tridecanoic acid (C13:0), myristic acid or tetradecanoic acid (C14:0), palmitic acid or hexadecanoic acid (C16:0), stearic acid or octodecanoic acid (C18:0), arachidic acid or eicosanoic acid (C20:0), behenic acid or docosanoic acid (C22:0) and lignoceric acid or tetracosanoic acid (C24:0).

The expression [symbol] unsaturated fatty acid having 12 to 24 carbon atoms [symbol] designates a monounsaturated or polyunsaturated fatty acid.

The monounsaturated straight-chain fatty acids having 12 to 24 carbon atoms are the acids of the following general formulas:

H₂C═CH—(CH₂)ₚ—COOH wherein p varies from 9 to 21;

H₃C—(CH₂)ₙ—HC═CH—(CH₂)ₚ—COOH wherein n and p vary from 0 to 20 and n+p vary from 8 to 20.

The stereochemistry of each unsaturation may be cis or trans.

Examples of straight-chain monounsaturated fatty acids having 12 to 24 carbon atoms, these not being limiting, are the following:

lauroleic acid or cis-9-dodecanoic acid (C12:1-w-3), oleic acid or cis-9-octadecenoic acid (C18:1-w-9) and selacholeic acid or cis-15-tetracoseonic acid (C24:1-w-9).

Examples of straight-chain polyunsaturated fatty acids having 12 to 24 carbon atoms, these not being limiting, are the following:

linoleic acid or cis-cis-9,12-octadecadienoic acid (C18:2-w-6), g-linoleic acid or cis-cis-cis-6,9,12-octadecatrienoic acid (C18:3-w-6) and arachidonic acid or cis-cis-cis-cis-5,8,11,14-icosatetraenoic acid (C20:4-w-6).

In one advantageous embodiment, the present invention concerns the use of an oil phase with eutectic invariant (PHE) such as defined above wherein the said fatty acid is selected from the following: lauric acid or dodecanoic acid, tridecylic acid or tridecanoic acid, myristic acid or tetradecanoic acid, palmitic acid or hexadecanoic acid, stearic acid or octodecanoic acid, arachidic acid or eicosanoic acid, behenic acid or docosanoic acid, lignoceric acid or tetracosanoic acid, lauroleic acid or cis-9-dodecanoic acid, selacholeic acid or cis-15-tetracoseonic acid g-linoleic acid or cis-cis-cis-6,9,12-octadecatriencic acid and arachidonic acid or cis-cis-cis-cis-5,8,11,14-icosatetraenoic acid.

In one advantageous embodiment, the present invention concerns the use of an oil phase with eutectic invariant (PHE) wherein the said fatty acid is selected from the following: lauric acid or dodecanoic acid, tridecylic acid or tridecanoic acid, myristic acid or tetradecanoic acid, palmitic acid or hexadecanoic acid, stearic acid or octodecanoic acid, arachidic acid or eicosanoic acid, behenic acid or docosanoic acid, lignoceric acid or tetracosanoic acid, lauroleic acid or cis-9-dodecanoic acid, selacholeic acid or cis-15-tetracoseonic acid, g-linoleic acid or cis-cis-cis-6,9,12-octadecatrienoic acid and arachidonic acid or icosatetraenoic acid such as defined above, and additionally comprising an unsaturated fatty acid such as defined above.

In one advantageous embodiment, the present invention concerns the use of an oil phase with eutectic invariant (PHE) such as defined above wherein the said local anaesthetic is lidocaine and the said fatty acid is lauric acid or tridecanoic acid (Example 1).

In this embodiment only one local anaesthetic is used: lidocaine.

One of the advantages of the invention and in particular of the lidocaine-lauric acid and lidocaine-tridecanoic acid mixtures is that they have a wide range of weight composition for which the mixture is a homogeneous liquid at 25° C. The diffusing properties of the local anaesthetic are slightly slower than for EMLA® but are more regular if the oil phase (PHE) is a mixture of lidocaine at 46% by weight and lauric acid (Example 2).

In one advantageous embodiment, the present invention concerns the use of an oil phase with eutectic invariant (PHE) such as defined above wherein the said oil phase is in homogeneous liquid form stable at a temperature of between 0° C. and 25° C., in particular between 0° C. and 10° C.

Another advantage of the invention is to be able to cold store the oil-in-water emulsions of the invention since the oil phase contained in the said emulsions at temperatures of between 0° C. and 10° C. remain homogeneous and liquid throughout the length of the authorized storage time for use of the said emulsion, which allows the preventing of possible oxidation of the oil phases at temperatures of between 25° C. and 37° C.

In one advantageous embodiment, the present invention concerns the use of an oil phase with eutectic invariant (PHE) such as defined above wherein the local, anaesthetic is lidocaine in a proportion of 50% by weight and the fatty acid is in a proportion of 50% by weight relative to the total weight of the oil phase (PHE), and the said oil phase is in homogeneous liquid form stable at a temperature of between 0° C. and 10° C.

In one advantageous embodiment the present invention concerns the use of an oil phase with eutectic invariant (PHE) such as defined above wherein the local anaesthetic is lidocaine in a proportion of % by weight and the fatty acid is lauric acid or tridecanoic acid in a proportion of 50% by weight relative to the total weight of the oil phase (PHE), and the said oil phase is in homogeneous liquid form stable at a temperature of between 0° C. and 10° C.

According to another aspect the present invention concerns an oil-in-water emulsion (O/W) comprising an oil phase with eutectic invariant (PHE) containing at least one local anaesthetic in a proportion of 10% to 60% by weight relative to the total weight, of the oil phase (PHE) and at least one fatty acid in a proportion of 40% to 90% by weight relative to the total weight of the oil phase (PHE), the said oil phase (PHE) being of weak ionic nature and in homogeneous liquid form stable at a temperature of between 0° C. and 37° C., provided that when the said local anaesthetic is mepivacaine or bupivacaine either the local anaesthetic is associated with at least one second local anaesthetic differing therefrom, or at least two different fatty acids are used to obtain a eutectic invariant such as defined above, and wherein the pH of the hydrophilic phase is between 6.5 and 7.5.

One of the advantages of the emulsions of the invention is that they have slower diffusion, which allows the obtaining of delayed effect or sustained effect properties.

Compared with EMLA which has rapidly decreasing diffusion kinetics, the emulsions of the invention have slower diffusion kinetics therefore decreasing at a slower rate.

In one advantageous embodiment, the present invention concerns an oil-in-water emulsion (O/W) such as defined above wherein the said oil phase (PHE) comprises at least one local anaesthetic in a proportion of 10% to 50% by weight relative to the total weight of the oil phase (PHE) and at least one fatty acid in a proportion of 50% to 90% by weight relative to the total weight of the oil phase (PHE).

In one advantageous embodiment, the present invention concerns an oil-in-water emulsion (O/W) such as defined above, wherein the said oil phase (PHE) comprises at least one local anaesthetic in a proportion of 20% to 60% by weight relative to the total weight of the oil phase (PHE), and at least one fatty acid in a proportion of 40% to 80% by weight relative to the total weight of the oil phase (PHE).

In one advantageous embodiment, the present invention concerns an oil-in-water emulsion (O/W) such as defined above wherein the said oil phase (PHE) comprises at least one local anaesthetic in a proportion of 30% to 60% by weight relative to the total weight of the oil phase (PHE), and at least one fatty acid in a proportion of 40% to 70% by weight relative to the total weight of the oil phase (PHE).

In one advantageous embodiment the present invention concerns an oil-in-water emulsion (O/W) such as defined above wherein the said oil phase (PHE) comprises at least one local anaesthetic in a proportion of 30% to 50% by weight relative to the total weight of the oil phase (PHE), and at least one fatty acid in a proportion of 60% to 70% by weight relative to the total weight of the oil phase (PHE).

In one advantageous embodiment the present invention concerns an oil-in-water emulsions (O/W) such as defined above wherein the said oil phase (PHE) comprises at least one local anaesthetic in a proportion of 40% to 50% by weight relative to the total weight of the oil phase (PHE), and at least one fatty acid in a proportion of 50% to 60% by weight relative to the total weight of the oil phase (PHE).

In one advantageous embodiment the present invention concerns an oil-in-mater emulsion (O/W) such as defined above wherein the said oil phase (PHE) comprises at least one local anaesthetic in a proportion of 40%, 41%, 42%, 43%, 44%, 45%, 46%, %, 4$ %, 49% or 50% by weight relative to the total weight of the oil phase (PHE), and at least one fatty acid in a proportion of 50%, 51%, 52%, 53%, 54%, 55%, 56%, 57%, 58%, 59% or 60% by weight relative to the total weight of the oil phase (PHE).

In one advantageous embodiment, the present invention concerns an oil-in-water emulsion (O/W) such as defined above wherein the said local anaesthetic is selected from among lidocaine, mepivacaine, bupivacaine, levobupicaine, prilocaine, articaine, ropivacaine, dibucaine and the mixtures thereof.

In one advantageous embodiment the present invention concerns an oil-in-water emulsion (O/W) such as defined above wherein the said local anaesthetic is selected from among lidocaine, levobupicaine, prilocaine, articaine, ropivacaine, dibucaine and the mixtures thereof.

In one advantageous embodiment the present invention concerns an oil-in-water emulsion (O/W) such as defined above wherein the said local anaesthetic is lidocaine.

In one advantageous embodiment, the present invention concerns an oil-in-water emulsion (O/W) such as defined above wherein the said local anaesthetic is lidocaine in a proportion of 50% relative to the total weight of the oil phase (PHE).

In one advantageous embodiment, the present invention concerns an oil-in-water emulsion (O/W) such as defined above wherein the said local anaesthetic is lidocaine in a proportion of 50% and the fatty acid is in a proportion of 50% by weight relative to the total weight of the oil phase (PHE).

In one advantageous embodiment, the present invention concerns an oil-in-water emulsion (O/W) such as defined above wherein the said local anaesthetic is lidocaine in a proportion of 50% and the fatty acid is in a proportion of 50% by weight relative to the total weight of the oil phase (PHE), the said oil phase being in homogeneous liquid form stable at a temperature of between 0° C. and 10° C.

In one advantageous embodiment, the present invention concerns an oil-in-water emulsion (O/W) such as defined above wherein the said fatty acid is a saturated or unsaturated, straight-chain or branched fatty acid having 12 to 24 carbon atoms.

In one advantageous embodiment, the present invention concerns an oil-in-water emulsion (O/W) such as defined above wherein the said fatty acid is selected from among the following: lauric acid or dodecanoic acid, tridecylic acid or tridecanoic acid, myristic acid or tetradecanoic acid, palmitic acid or hexadecanoic acid, stearic acid or octodecanoic acid, arachidic acid or eicosanoic acid, behenic acid or docosanoic acid, lignoceric or tetracosanoic acid, lauroleic acid or cis-9-dodecanoic acid, selacholeic acid or cis-15-tetracoseonic acid, g-linoleic acid or cis-cis-cis-6,9,12-octadecatrienoic acid and arachidonic acid or cis-cis-cis-cis-5,8,11,14-icosatetraenoic acid.

In one advantageous embodiment, the present invention concerns an oil-in-water emulsion (L/H) such as defined above wherein the said fatty acid is selected from among the following: lauric acid or dodecanoic acid, tridecylic acid or tridecanoic acid, myristic acid or tetradecanoic acid, palmitic acid or hexadecanoic acid, stearic acid or octodecanoic acid, arachidic acid or eicosanoic acid, behenic acid or docosanoic acid, lignoceric acid or tetracosanoic acid, lauroleic acid or cis-9-dodecanoic acid, selacholeic acid or cis-15-tetracoseonic acid, g-linoleic acid or cis-cis-cis-6,9,12-octadecatrienoic acid and arachidonic acid or cis-cis-cis-cis-5,8,11,14-icosatetraenoic acid such as defined above, and additionally comprising an unsaturated fatty acid such as defined above.

In one advantageous embodiment, the present invention concerns an oil-in-water emulsion (O/W) such, as defined above wherein the said oil phase (PHE) is selected from among the following oil phases (PHE): lidocaine-lauric acid or lidocaine-tridecanoic acid.

In one advantageous embodiment, the present invention concerns an oil-in-water emulsion (O/W) such as defined above, the said oil phase with eutectic invariant (PHE) being in a proportion relative to the total weight of the emulsion of 5% to 25% by weight, preferably 10% to 25% by weight.

In one advantageous embodiment, the present invention concerns an oil-in-water emulsion (O/W) comprising an oil phase with eutectic invariant (PHE) such as defined above, wherein the oil phase is dispersed in a hydrophilic phase including one or more thickeners and one or more emulsifiers.

The term [symbol] thickeners [symbol] designates all thickeners which can be used to prepare an emulsion and are well known to those skilled in the art.

Preferred thickeners are the following: cellulose and derivatives (methyl and carbomethyl cellulose), alginates, gelatine, anionic polymers, gums (arabic, tragacanth), colloidal silica, wool grease, beeswax.

The term [symbol] emulsifiers [symbol] designates all emulsifiers and self-emulsifiers which can be used to prepare an emulsion and are well known to persons skilled in the art.

Preferred emulsifiers are the following: non-ionic emulsifiers with ester bond (sorbitan esters, polysorbates), with ether bond (fatty alcohol ethers and PEG ethers), lecithin, glycerol, glycerol and fatty acid esters (triglycerides).

In one advantageous embodiment the present invention concerns an oil-in-water emulsion (O/W) such as defined above comprising:
5 to 20 weight % of an oil phase with eutectic invariant (PHE) such as defined above, in particular 15%;
30 to 40 weight % of glycerol;
5 to 15 weight % of polysorbate, in particular polysorbate 85; and
22 to 60 weight % of a hydrophilic phase, optionally thickened with 0 to 3 weight % of a cellulose derivative.

Glycerol is used herein as self-emulsifier and the emulsion therefore corresponds to a self-emulsion.

The term [symbol] self-emulsifier [symbol] designates emulsification which occurs spontaneously, in other words it designates any agent or composition capable of forming a stable emulsion with an aqueous phase with practically no supply of energy (i.e. without the need for applying thermal and/or mechanical energy) e.g. by dispersion in the aqueous phase via slow mechanical agitation, also called a self-emulsion.

The emulsions described in this embodiment are therefore emulsions having self-emulsifying properties i.e. if, after a certain storage time e.g. 15 days in a container the emulsion has become de mixed, it is sufficient to shake manually the said container to re-form the said emulsion inside the said container without having to apply strong agitation or activating energy.

They also have the advantage of having a smaller vesicle size than conventional emulsions i.e. smaller than 1 μm, whereas in conventional emulsions it is larger than 1 μm.

The emulsion in this embodiment is preferably intended for an analgesic application.

In one advantageous embodiment, the present invention concerns an oil-in-water emulsion (O/W) such as defined above comprising:
5 to 20 weight % of an oil phase with eutectic invariant (PHE) such as defined above, in particular 10%;
1 to 3 weight % of lecithin, in particular egg or soy;
74 to 93 weight % of a hydrophilic phase, particularly thickened with 1 to 3 weight % of a cellulose derivative.

Glycerol is used here as self-emulsifier and the emulsion therefore corresponds to a self-emulsion.

The emulsion in this embodiment is preferably intended for antalgic or sexual retardant application.

In one advantageous embodiment, the present invention concerns an oil-in-water emulsion (C7W) such as defined above comprising:
5 to 20 weight % of an oil phase with eutectic invariant (PHE) such as defined above, in particular 15%;
40 to 50 weight % of glycerol;
5 to 15 weight % of polysorbate, in particular polysorbate 85; and
22 to 60 weight % of a hydrophilic phase optionally thickened with 0.5 to 3 weight % of an alginate.

Glycerol is used here as self-emulsifier and the emulsion therefore corresponds to a self-emulsion.

The emulsion in this embodiment is preferably intended for antalgic or sexual retardant application.

In one advantageous embodiment, the present invention concerns an oil-in-water emulsion (O/W) such as defined above comprising:
5 to 20 weight % of an oil phase with eutectic invariant (PHE) such as defined above, in particular 15%;
40 to 50 weight % of glycerol;
5 to 15 weight % of polysorbate, in particular polysorbate 85; and
22 to 60 weight % of a hydrophilic phase optionally thickened with 0.5 to 3 weight % of gelatine.

The emulsion in this embodiment is preferably intended for antalgic or sexual retardant application.

In one advantageous embodiment, the present invention concerns an oil-in-water emulsion (O/W) such as defined above, comprising:
5 to 20% by weight of an oil phase with eutectic invariant (PHE) such as defined above, in particular 15%;
40 to 50 weight % of glycerol;
5 to 15 weight % of polysorbate, in particular polysorbate 85; and
22 to 60 weight % of a hydrophilic phase optionally thickened with 0.5 to 3 weight % of an anionic polymer.

The emulsion in this embodiment is preferably intended for antalgic or sexual retardant application.

In one advantageous embodiment the present invention concerns an oil-in-water emulsion (O/W) such as defined above additionally comprising a vegetable oil or synthetic short-, medium- or long-chain triglyceride oil.

By the expression [symbol] vegetable oil or synthetic short-chain triglyceride oil [symbol] is to be understood short-chain fatty acids (<8 carbon atoms) such as propionic acid, butyric acid.

By the expression [symbol] vegetable oil or synthetic medium-chain triglyceride oil [symbol] is meant medium-chain fatty acids (8 to 12 carbon atoms) such as caprylic, capric, lauric acids and copra oil.

By the expression [symbol] vegetable oil or synthetic long-chain triglyceride oil [symbol] is meant long-chain fatty acids (>C12) such as palmitic, oleic, linoleic, linolenic acids and olive, soy, corn, groundnut and sunflower oils.

In one advantageous embodiment, the present invention concerns an oil-in-water emulsion (O/W) additionally comprising a vegetable oil or synthetic short-, medium- or long-chain triglyceride oil such as defined above, wherein the said triglyceride oil is a medium-chain triglyceride oil, the said medium-chain triglyceride oil being present in particular up to 15% by weight.

In one advantageous embodiment of the invention the oil phase (PHE) of the oil-in-water emulsion (O/W) such as previously defined is formed of 50% by weight of lidocaine and 50% by weight of lauric acid.

In one advantageous embodiment of the invention the oil phase (PHE) of the oil-in-water emulsion (O/W) such as previously defined is formed of 50% by weight of lidocaine and 50% by weight of tridecanoic acid.

According to another aspect the present invention concerns a pharmaceutical composition comprising an oil-in-water emulsion (O/W) comprising an oil phase with eutectic invariant (PHE) such as defined above in association with a pharmaceutically acceptable vehicle.

By [symbol] pharmaceutically acceptable vehicle [symbol] is meant a carrier allowing the conveying of the oil phase and which is not an excipient. Non-limiting examples of carriers are cream, gel and patch.

In one advantageous embodiment the present invention concerns a pharmaceutical composition in association with a pharmaceutically acceptable vehicle such as defined above in a form which can be administered via topical route at a dose of 0.5 g/10 cm² to 4 g/10 cm².

According to another aspect the present invention concerns an oil-in-water emulsion comprising an oil phase such as defined above for use thereof as medicinal product in particular as analgesic, antalgic or sexual retardant.

Through the presence of the oil phase with high anaesthetic content the said emulsion via topical application to the skin allows diffusion of the anaesthetic through the Stratum Corneum which is the main physiological barrier of human skin.

One another advantage of the invention is that the oil phase with eutectic invariant comprises a fatty acid devoid of toxicity which acts as promoter of transdermal diffusion of the anaesthetic.

The term [symbol] antalgic [symbol] refers to a medicinal product intended to reduce pain.

The term [symbol] sexual retardant [symbol] refers to medicinal product slowing the onset of ejaculation.

The slower diffusion capability of the emulsions of the invention imparts delayed effect properties thereto or a sustained effect as indicated above, these properties being of particular interest for pain alleviation or analgesia.

In one advantageous embodiment the present invention concerns an oil-in-water emulsion comprising an oil phase such as defined above for use thereof as medicinal product, such as defined above, in particular as analgesic.

In one advantageous embodiment, the present invention concerns an oil-in-water emulsion comprising an oil phase such as defined above for use thereof as medicinal product, such as defined above, in particular as antalgic.

In one advantageous embodiment the present invention concerns an oil-in-water emulsion comprising an oil phase such as defined above for use thereof as medicinal product, such as defined above, in particular as sexual retardant.

According to another aspect the present invention concerns an oil phase with eutectic invariant (PHE) comprising at least one local anaesthetic in a proportion of 10% to 60% by weight relative to the total weight of the oil phase (PHE), and at least one fatty acid in a proportion of 40% to 90% by weight relative to the total weight of the oil phase (PHE) to form an oil-in-water emulsion (O/W) wherein the pH of the hydrophilic phase is between 6.5 and 7.5, the said oil phase (PHE) being of weak ionic nature and in homogeneous liquid form that is stable at a temperature of between 0° C. and 37° C., provided that when the said local anaesthetic is mepivacaine or bupivacaine it is associated with a local anaesthetic differing therefrom or with at least two different fatty acids to obtain a eutectic invariant

wherein the said local anaesthetic is selected from among lidocaine, mepivacaine, bupivacaine, levobupicaine, prilocaine, articaine, ropivacaine, dibucaine and the mixtures thereof, and the said fatty acid is selected from among the following: lauric acid or dodecanoic acid, tridecylic acid or tridecanoic acid, myristic acid or tetradecanoic acid, palmitic acid or hexadecanoic acid, stearic acid or octodecanoic acid, arachidic acid or eicosanoic acid, behenic acid or docosanoic acid, lignoceric acid or tetracosanoic acid, lauroleic acid or cis-9-dodecanoic acid, selacholeic acid or cis-15-tetracoseonic acid, g-linoleic acid or cis-cis-cis-6,9,12-octadecatrienoic acid and arachidonic acid or cis-cis-cis-cis-5,8,11,14-icosatetraenoic acid.

According to another aspect, the present invention concerns an oil phase with eutectic invariant (PHE) comprising at least one local anaesthetic in a proportion of 10% to 60% by weight relative to the total weight of the oil phase (PHE), and at least one fatty acid in a proportion of 40% to 90% by weight relative to the total weight of the oil phase (PHE), to form an oil-in-water emulsion (O/W) wherein the pH of the hydrophilic phase is between 6.5 and 7.5, the said oil phase (PHE) being of weak ionic nature and in homogeneous liquid form that is stable at a temperature of between 0° C. and 37° C., provided that when the said local anaesthetic is mepivacaine or bupivacaine, it is associated either with a local anaesthetic differing therefrom or with at least two different fatty acids, to obtain a eutectic invariant,

wherein the said local anaesthetic is chosen from among lidocaine, levobupicaine, prilocaine, articaine, ropivacaine, dibucaine and the mixtures thereof, and the said fatty acid is selected from among the following: lauric acid or dodecanoic acid, tridecylic acid or tridecanoic acid, myristic acid or tetradecanoic acid, palmitic acid or hexadecanoic acid, stearic acid or octodecanoic acid, arachidic acid or eicosanoic acid, behenic acid or docosanoic acid, lignoceric acid or tetracosanoic acid, lauroleic acid or cis-9-dodecanoic acid, selacholeic acid or cis-15-tetracoseonic acid, g-linoleic acid or cis-cis-cis-6,9,12-octadecatrienoic acid and arachidonic acid or cis-cis-cis-cis-5,8,11,14-icosatetraenoic acid.

In one advantageous embodiment the present invention concerns an oil phase with eutectic invariant (PHE) such as defined above wherein the said local anaesthetic is in a proportion of 10% to 50% by weight relative to the total weight of the oil phase (PHE) and the said fatty acid is in a proportion of 60% to 90% by weight relative to the total weight of the oil phase (PHE).

In one advantageous embodiment the present invention concerns an oil phase with eutectic invariant (PHE) such as defined above wherein the said local anaesthetic is in a proportion of 20% to 60% by weight relative to the total weight of the oil phase (PHE) and the said fatty acid is in a proportion of 40% to 80% by weight relative to the total weight of the oil phase (PHE).

In one advantageous embodiment, the present invention concerns an oil phase with eutectic invariant (PHE) such as defined above wherein the said local anaesthetic is in a proportion of 30% to 60% by weight relative to the total weight of the oil phase (PHE) and the said fatty acid is in a proportion of 40% to 70% by weight relative to the total weight of the oil phase (PHE).

In one advantageous embodiment the present invention concerns an oil phase with eutectic invariant (PHE) such as defined above wherein the said local anaesthetic is in a proportion of 30% to 50% by weight relative to the total weight of the oil phase (PHE) and the said fatty acid is in a proportion of 50% to 70% by weight relative to the total weight of the oil phase (PHE).

In one advantageous embodiment, the present invention concerns an oil phase with eutectic invariant (PHE) such as defined above wherein the said local anaesthetic is in a proportion of 40% to 50% by weight and the said fatty acid in a proportion of 50% to 60% by weight, the said oil phase with eutectic invariant being in the form of a homogeneous liquid that is stable at a temperature of between 0° C. and 37° C., in particular between 0° C. and 10° C.

In one advantageous embodiment, the present invention concerns an oil phase with eutectic invariant (PHE) such as defined above wherein the said local anaesthetic is in a proportion of 41%, 42%, 43%, 44%, 45%, 46%, 47%, 48%, 49% or 50% by weight and the said fatty acid in a proportion of 50%, 51%, 52%, 53%, 54%, 55%, 56%, 57%, 58%, 59% or 60% by weight.

In one advantageous embodiment the present invention concerns an oil phase with eutectic invariant (PHE) such as defined above wherein the said local anaesthetic is lidocaine and the said fatty acid is chosen from among lauric acid or tridecanoic acid.

In one advantageous embodiment the present invention concerns an oil phase with eutectic invariant (PHE) such as defined above wherein the said local anaesthetic is lidocaine in a proportion of 50% and the said fatty acid is chosen from among lauric acid or tridecanoic acid in a proportion of 50%.

In one advantageous embodiment the present invention concerns an oil phase with eutectic invariant (PHE) such as defined above wherein the said local anaesthetic is lidocaine in a proportion of 50% and the said fatty acid is lauric acid in a proportion of 50%, the said oil phase with eutectic invariant being in the form of a homogenous liquid that is stable at a temperature of between 0° C. and 10° C.

### DESCRIPTION OF THE FIGURES

FIG. 1: Diagram of liquid-solid phases of a binary system with eutectic invariant consisting of lidocaine and lauric acid, determined by differential scanning calorimetry.

X-axis: weight percentage of lidocaine in the lidocaine-lauric acid mixture.

Y-axis: T(° C.) is the temperature expressed in degrees Celsius.

FIG. 2: Diagram of liquid-solid phases of a binary system with eutectic invariant consisting of lidocaine and tridecanoic acid, determined by differential scanning calorimetry.

X-axis: lidocaine percentage in the lidocaine-tridecanoic acid mixture.

Y-axis: T (° C.) is the temperature expressed in degrees Celsius.

FIG. 3: Diffusion of anaesthetics in the mixtures with eutectic invariant (oil phases) over a period of 6 hours.

X-axis: t(h) is the time expressed in hours.

Y-axis: n/mol

For periods of 1, 2, 4, 6 hours, the solid circles (from top to bottom) correspond to:
lidocaine in the lidocaine-prilocaine mixture
prilocaine in the lidocaine-prilocaine mixture
lidocaine in the lidocaine-lauric acid mixture according to the invention.

FIGS. 4A and B: Self-emulsion of Example 5 including a lidocaine-lauric acid mixture

FIG. 4A: de-mixed self-emulsion. FIG. 4A clearly shows two phases.

FIG. 4B: after manual shaking of the de-mixed self-emulsion. The composition is in emulsion form and only shows a single phase.

FIG. 5: Diffusion of anaesthetics in the emulsion of Example 5 and in MLA over a period of 6 hours.

X-axis: t(h) is time expressed in hours.

Y-axis: n/mol.

For a period of 1 hour the solid circles (from top to bottom) correspond to:
lidocaine in EMLA cream
prilocaine in EMLA cream
lidocaine in the emulsion of the invention.

For the periods of 2 and 6 hours the solid circles (from top to bottom) correspond to:
prilocaine in the lidocaine-prilocaine mixture (EMLA)
lidocaine in the lidocaine-prilocaine mixture (EMLA)
lidocaine in the emulsion of the invention.

At times 0 and 4 hours all the circles merge.

The diffusion of the anaesthetic is slower with the emulsion of the invention which, in antalgic and sexual retardant applications in particular, allows an effect to be obtained that is attenuated but better distributed over time and hence better than EMLA.

Also the use of fatty acids present in the skin allows a formulation to be obtained that is adapted for topical use and is additionally free of prilocaine.

### EXAMPLES

### Example 1

### Preparation of Oil Phases with Eutectic Invariant Comprising a Local Anaesthetic and a Fatty Acid

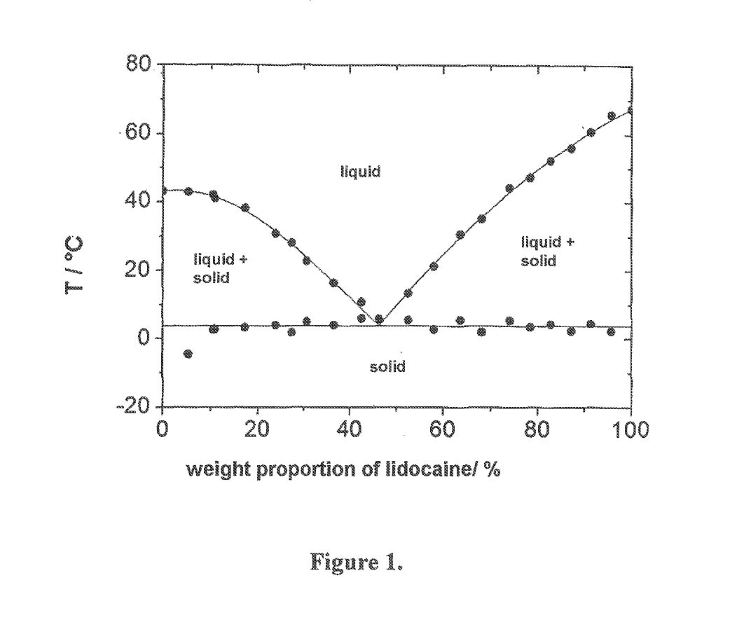

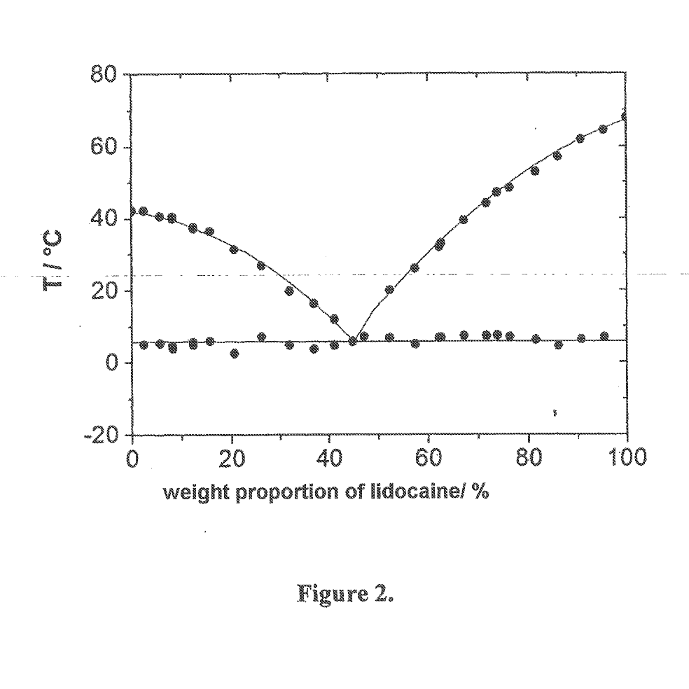

The mixing at ambient temperature in a container of a local anaesthetic and of a fatty acid, both initially solid, leads to partial or full spontaneous melting of the two compounds into a homogeneous liquid. Full melting is obtained via the suitable choice of anaesthetic, fatty acid and their relative proportion in the mixture. The binary mixtures of lidocaine with lauric acid and tridecanoic acid exhibit this property. The phase diagrams of the mixtures of lidocaine with lauric acid and tridecanoic acid determined by differential scanning calorimetry are respectively given in FIGS. 1 and 2. The properties of the binary mixtures of lidocaine-fatty acids are given, in Table I opposite the properties of the binary lidocaine-prilocaine mixture (EMLA). The binary lidocaine-fatty acid mixtures have improved thermodynamic properties compared with the binary lidocaine-prilocaine mixture (EMLA) i.e. a lower melt point and wider composition range over which the binary mixture is liquid at ambient temperature (25° C.) and at low temperature (10° C.) Table I gives the characteristic thermodynamic properties of the eutectic mixtures of lidocaine-lauric acid and lidocaine-tridecanoic acid conforming to the invention and of the eutectic lidocaine-prilocaine mixture (EMLA) as comparison.

TABLE I

| | Eutectic | eutectic | composition weight range | composition weight range |
|---|---|---|---|---|
| | melting | weight | over which the binary | over which the binary |
| Binary | temperature | composition | mixture is liquid at | mixture is liquid at |
| mixture | (° C.) | (% O) | 25° C. (%) | 10° C. (%) |
| lidocaine-lauric | 6 | 46 | 31-60 | 42-49 |
| acid (L-C12) | | | | |
| lidocaine- | 6 | 47 | 28-57 | 42-46 |
| tridecanoic acid | | | | |
| (L-C13) | | | | |
| lidocaine- | 18 | 50 | 48-61 | none |
| prilocaine(EMLA) | | | | |

The percentage values are expressed with ±2% error i.e. for L-C12 for example the range varies from 31 ± 2 to 60 ± 2.

### Example 2

### Diffusion Kinetics Through a Lipophilic Membrane of Lidocaine in an Oil Phase with Eutectic Invariant (PHE) Having a Composition of 50 Weight % Lidocaine and 50 Weight % Lauric Acid

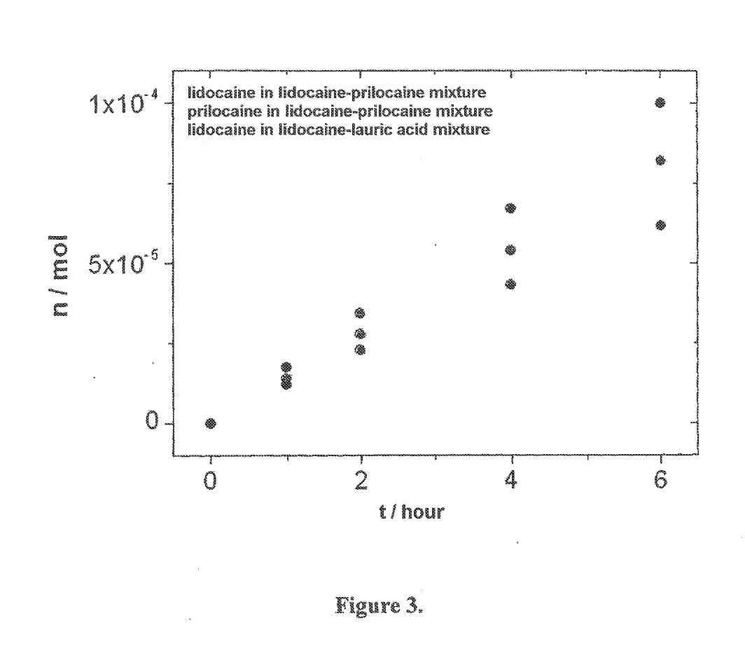

The diffusion kinetics of lidocaine contained in the mixture of 50% lidocaine by weight and 50% lauric acid by weight, were measured using a Franz diffusion cell. The diffusion kinetics of lidocaine and prilocaine in the mixture of 50% lidocaine by weight and 50% prilocaine by weight, which corresponds to the eutectic oil phase of EMLA cream, were measured under the same conditions for comparison. The diffusion kinetics of the anaesthetics of these two mixtures are given in FIG. 3. The experimental conditions were the following: the diffusion membrane was a silicone film of 125 μm thickness, the surface area of the diffusion membrane was 3.1 cm²; the reservoir solution in the Franz cell was an aqueous buffer solution of pH 6.2, this value being close to the physiological pH of human skin; the Franz cell reservoir was thermostat-controlled at 37° C., the temperature of the human body.

### Example 3

### Emulsion Containing Triglycerides, Egg Lecithin and Cellulose

Oil-in-water emulsion (O/W) consisting of:
10 weight % of an oil phase (PHE) formed of 50 weight % lidocaine and 50 weight % lauric acid;
10 weight % of medium-chain triglyceride oil;
1.2 weight % of egg lecithin dispersed in 78.3 weight % of a hydrophilic phase thickened with 0.5 weight % methylcellulose.

The lecithin was dispersed in the medium-chain triglyceride oil brought to a temperature of 60° C. The methylcellulose was dispersed at 25° C. in the aqueous phase. Emulsification was performed at 25° C. using the phase inversion method with a disperser at a rate of 13000 rpm for 10 minutes. The coarse emulsion thus obtained was homogenized using an ultrasound homogenizer for 10 minutes. The emulsion obtained had a mean diameter of 100 nm with polydispersity index of 0.1 and zeta potential of −50 mV.

### Example 4

### Emulsion Containing Glycerol and Polysorbate

Oil-in-water (O/W) emulsion consisting of
15 weight % of an oil, phase (PHE) formed of 50 weight % lidocaine and 50 weight % lauric acid;
40 weight % glycerol;
5 weight % polysorbate 85; and
40 weight % of water.

The production method was performed by self-emulsification at 25° C. and was followed by homogenization using a homogenizer for 10 minutes. The mean diameter of the emulsion obtained was 140 nm, the polydispersity index 0.3 and zeta potential −50 mV.

### Example 5

### Self-Emulsion Including a Lidocaine-Lauric Acid Mixture Thickened with Polysorbate

A self-emulsion including an oil phase with eutectic invariant of lidocaine and lauric acid was formulated. This type of emulsion does not require heating or vigorous mechanical agitation to be formed. The weight composition of this self-emulsion was the following:

| | |
|---|---|
| polysorbate 85 | 5% |
| glycerol | 43% |
| water | 40% |
| lidocaine | 6% |
| lauric acid | 6% |

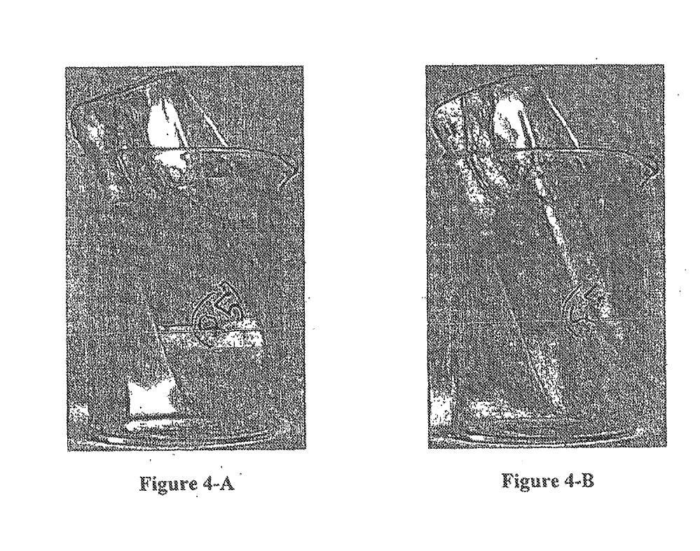

The mixture of these compounds is given in the foregoing (FIG. 4-A) and after manual shaking (FIG. 4-B) a white emulsion was formed from an initial mixture having lower hydrophilic phase separated from the upper lipophilic phase.

### Example 6

### Diffusion Kinetics Through a Lipophilic Membrane of Lidocaine Contained in an Emulsion Including an Oil Phase with Eutectic Invariant (PHE) Having a Composition of 50 Weight % Lidocaine and 50 Weight % Lauric Acid

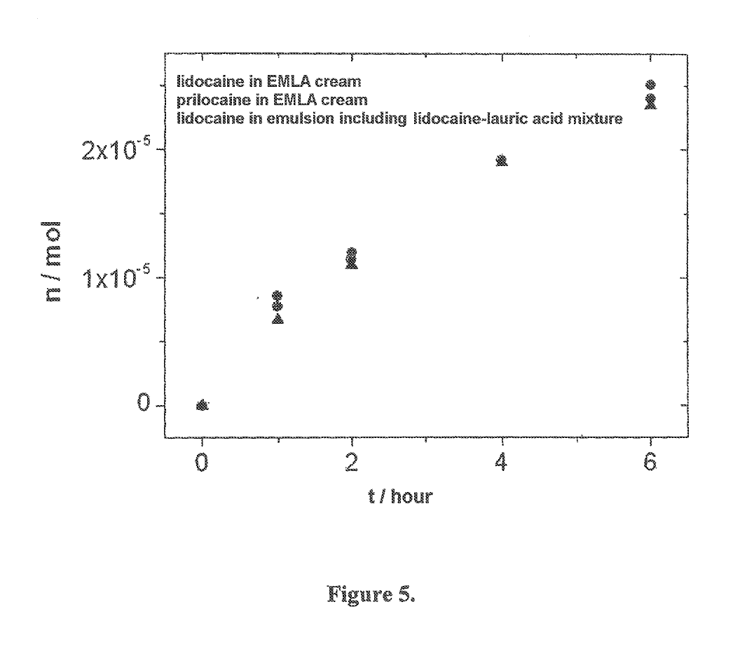

The diffusion kinetics, through a lipophilic membrane, of the lidocaine contained in an emulsion including an oil phase with eutectic invariant (PHE) having a composition of 50 weight % lidocaine and 50 weight % acid, were measured using a Franz diffusion cell. The diffusion kinetics of lidocaine and prilocaine in a generic of EMLA cream were measured under the same conditions for comparison. The diffusion kinetics of the anaesthetics contained in the emulsion of lidocaine, fatty acid and the self-emulsion, and of the EMLA cream are given in FIG. 5. The experimental conditions of these diffusion measurements are described in Example 2.

### Example 7

### Self-Emulsion Including a Lidocaine-Lauric Acid Mixture

A self-emulsion including an oil phase with eutectic invariant of lidocaine and lauric acid was formulated. This type of emulsion does not require heating or vigorous mechanical agitation to be formed. The weight composition of the self-emulsion was the following:

| | |
|---|---|
| medium-chain triglycerides | 8% |
| lidocaine | 6% |
| lauric acid | 6% |
| lecithin E80 | 1.2% |
| methylcellulose | 0.5% |
| water | 78.3% |

The manual shaking of these compounds is sufficient to form the self-emulsion.

### Example 8

### Nanoemulsions Including a Lidocaine-Lauric Acid Mixture and Sodium Alginate as Thickener

A nanoemulsion is an emulsion in which the size of some globules is in the order of one nanometre. A nanoemulsion including an oil phase with eutectic invariant of lidocaine and lauric acid with sodium alginate as thickener was formulated. This emulsion, was also a self-emulsion which can be formed by mere manual shaking of its constituents and without heating. The weight compositions of this nanoemulsion are given below. The Dv0.5 value which is the maximum diameter of the globules representing 50% of the population was measured over 3 days (Table 11) after formulation of the emulsion. This maximum diameter was less than 1 μm.

### Composition

| | |
|---|---|
| Polysorbate 85 | 5% |
| Glycerol | 43% |
| Water | 39% |
| Lidocaine | 6% |
| Lauric acid | 6% |
| Sodium alginate | 1% |

The pH of the emulsion was adjusted to 7.0 with sodium hydroxide

TABLE II
Trend in Dv0.5 of the emulsion

| Day | 0 | 1 | 3 |
|---|---|---|---|
| DvO.5 (nm) | 280 | 340 | 390 |

### Example 9

### Nanoemulsion Including a Lidocaine-Lauric Acid Mixture and Carbopol 981 as Thickener

A nanoemulsion including an oil phase with eutectic invariant of lidocaine and lauric acid with carbopol 981 as thickener was formulated. This emulsion is also a self-emulsion which can be formed by mere manual shaking of its constituents and without heating. The weight composition of this nanoemulsion is given below. The Dv0.5 was measured over 7 days after the formulation of the emulsion (Table III). This maximum diameter was less than 1 μm.

### Weight Composition

| | |
|---|---|
| PS85 | 5% |
| Glycerol | 43% |
| Water | 39% |
| Lidocaine | 6% |
| Lauric acid | 6% |
| Carbopol 981 | 1% |

The pH of the emulsion was adjusted to 6.7 with sodium hydroxide

TABLE III
Trend in Dv0.5 of the emulsion

| Day | 0 | 1 | 3 | 7 |
|---|---|---|---|---|
| Dv0.5 (nm) | 350 | 460 | 400 | 400 |

1. A method for forming an oil-in-water emulsion (O/W) using an oil phase with eutectic invariant (PHE) comprising at least one local anaesthetic in a proportion of 10% to 60% by weight relative to the total weight of the oil phase (PHE) and at least one fatty acid in a proportion of 40% to 90% by weight relative to the total weight of the oil phase (PHE),
wherein the pH of the hydrophilic phase is particularly between 6 and 8,
the said oil phase (PHE) being in homogeneous liquid form and stable at a temperature of between 0° C. and 37° C.,
provided that if the said local anaesthetic is mepivacaine or bupivacaine either the said local anaesthetic is associated with at least one second local anaesthetic differing therefrom, or at least two different fatty acids are used to obtain a eutectic invariant.

2. The method of claim 1 wherein the local anaesthetic is in a proportion of 30% to 60% by weight relative to the total weight of the oil phase (PHE) and the fatty acid is in a proportion of 40% to 70% by weight relative to the total weight of the oil phase (PHE).

3. The method of claim 1 wherein the local anaesthetic is in a proportion of 30% to 50% by weight relative to the total weight of the oil phase (PHE) and the fatty acid is in a proportion of 50% to 70% by weight relative to the total weight of the oil phase (PHE).

4. The method of claim 1 wherein the local anaesthetic is selected from among lidocaine, levobupicaine, prilocaine, articaine, ropivacaine, dibucaine and the mixtures thereof.

5. The method of claim 1 wherein the local anaesthetic is lidocaine.

6. The method of claim 1 wherein the said fatty acid is a saturated or unsaturated, straight-chain or branched fatty acid having 12 to 24 carbon atoms.

7. The method of claim 1 wherein the said fatty acid is selected from among the following: lauric acid or dodecanoic acid, tridecylic acid or tridecanoic acid, myristic acid or tetradecanoic acid, palmitic acid or hexadecanoic acid, stearic acid or octodecanoic acid, arachidic acid or eicosanoic acid, behenic acid or docosanoic acid, lignoceric acid or tetracosanoic acid, lauroleic acid or cis-9-dodecanoic acid, selacholeic acid or cis-15-tetracoseonic acid, g-linoleic acid or cis-cis-cis-6,9,12-acid and arachidonic acid or cis-cis-cis-cis-5,8,11,14-icosatetraenoic acid.

8. The method of claim 1 wherein the said local anaesthetic is lidocaine and the said fatty acid is lauric acid or tridecanoic acid.

9. The method of claim 1 wherein the said oil phase is in homogeneous liquid form and stable at a temperature of between 0° C. and 25° C.

10. An oil-in-water emulsion (O/W) comprising an oil phase with eutectic invariant (PHE) containing at least one local anaesthetic in a proportion of 10% to 60% by weight relative to the total weight of the oil phase (PHE) and at least one fatty acid in a proportion of 40% to 90% by weight relative to the total weight of the oil phase (PHE) as defined in claim 1, and wherein the pH of the hydrophilic phase is between 6.5 and 7.5.

11. The oil-in-water emulsion (O/W) of claim 10 wherein the said oil phase (PHE) comprises at least one local anaesthetic in a proportion of 30% to 60% by weight relative to the total weight of the oil phase (PHE) and at least one fatty acid in a proportion of 40% to 70% by weight relative to the total weight of the oil phase (PHE).

12. The oil-in-water emulsion (O/W) of claim 10 wherein the said oil phase (PHE) comprises at least one local anaesthetic in a proportion of 30% to 60% by weight relative to the total weight of the oil phase (PHE) and at least one fatty acid in a proportion of 40% to 70% by weight relative to the total weight of the oil phase (PHE).

13. The oil-in-water emulsion (O/W) of claim 10 wherein the said local anaesthetic is selected from among lidocaine, mepivacaine, bupivacaine, levobupicaine, prilocaine, articaine, ropivacaine, dibucaine and the mixtures thereof.

14. The oil-in-water emulsion (O/W) of claim 10 wherein the said local anaesthetic is lidocaine.

15. The oil-in-water emulsion (O/W) of claim 10 wherein the said fatty acid is a saturated or unsaturated, straight-chain or branched fatty acid having 12 to 24 carbon atoms.

16. The oil-in-water emulsion (O/W) of claim 10 wherein the said oil phase (PHE) is selected from among the following oil phases (PHE): lidocaine-lauric acid or lidocaine-tridecanoic acid.

17. The oil-in-water emulsion (O/W) of claim 10 wherein the said oil phase (PHE) is in a proportion relative to the total weight of the emulsion of 5% to 25% by weight.

18. The oil-in-water emulsion (O/W) of claim 10 wherein the said oil phase is dispersed in a hydrophilic phase including one or more thickeners and one or more emulsifiers.

19. The oil-in-water emulsion (O/W) of claim 10 comprising:
5 to 20 weight % of an oil phase with eutectic invariant (PHE), in particular 15%;
30 to 40 weight % of glycerol;
5 to 15 weight % of polysorbate, in particular polysorbate 85; and
22 to 60 weight % of hydrophilic phase optionally thickened with 0 to 3 weight % of a cellulose derivative.

20. The oil-in-water emulsion (O/W) of claim 10 comprising:
5 to 20 weight % of an oil phase with eutectic invariant (PHE), in particular 10%;
1 to 3 weight % of lecithin, in particular egg or soy lecithin;
74 to 93 weight % of hydrophilic phase, particularly thickened with 1 to 3 weight % of a cellulose derivative.

21. The oil-in-water emulsion (O/W) of claim 10 comprising:
5 to 20 weight % of an oil phase with eutectic invariant (PHE) such as defined above, in particular 15%;
40 to 50 weight % of glycerol;
5 to 15 weight % of polysorbate, in particular polysorbate 85;
22 to 60 weight % of hydrophilic phase optionally thickened with 0.5 to 3 weight % of alginate.

22. The oil-in-water emulsion (O/W) of claim 10 comprising
5 to 20 weight % of an oil phase with eutectic invariant (PHE) such as defined above, in particular 15%;
40 to 50 weight % of glycerol;
5 to 15 weight % of polysorbate, in particular polysorbate 85; and
22 to 60 weight % of hydrophilic phase optionally thickened with 0.5 to 3 weight % of gelatine.

23. The oil-in-water emulsion (O/W) of claim 10 comprising:
5 to 20 weight % of an oil phase with eutectic invariant (PHE) such as defined above, in particular 15%;
40 to 50 weight % of glycerol;
5 to 15 weight % of polysorbate, in particular polysorbate 85; and
22 to 60 weight % of hydrophilic phase optionally thickened with 0.5 to 3 weight % of an anionic polymer.

24. The oil-in-water emulsion (O/W) of claim 10 additionally comprising a vegetable oil or synthetic short-, medium- or long-chain triglyceride oil.

25. The oil-in-water emulsion (O/W) of claim 24 wherein the said triglyceride oil is a medium-chain triglyceride oil, the said medium-chain triglyceride oil being present in particular up to 15% by weight.

26. The oil-in-water emulsion (O/W) of claim 10 wherein the oil phase (PHE) is formed of 50% by weight of lidocaine and 50% by weight of lauric acid.

27. A pharmaceutical composition comprising an oil-in-water emulsion (O/W) according to claim 10 in association with a pharmaceutically acceptable vehicle.

28. A method for treating or alleviating pain, or for delaying ejaculation, comprising administering an oil-in-water emulsion comprising an oil phase as defined in claim 10 to a patient in need thereof.

29. The oil phase (PHE) as defined in claim 1 wherein the said local anaesthetic is selected from among lidocaine, mepivacaine, bupivacaine, levobupivacaine, prilocaine, articaine, ropivacaine, dibucaine and the mixtures thereof, and the said fatty acid is selected from among the following: lauric acid or dodecanoic acid, tridecylic acid or tridecanoic acid, myristic acid or tetradecanoic acid, palmitic acid or hexadecanoic acid, stearic acid or octodecanoic acid, arachidic acid or eicosanoic acid, behenic acid or docosanoic acid, lignoceric acid or tetracosanoic acid, lauroleic acid or cis-9-dodecanoic acid, selacholeic acid or cis-15-tetracoseonic acid, g-linoleic acid or cis-cis-cis-6,9,12-octadecatrienoic acid and arachidonic acid or cis-cis-cis-cis-5,8,11,14-icosatetraenoic acid.

30. The oil phase (PHE) as defined in claim 1 wherein the said local anaesthetic is selected from among lidocaine, levobupicaine, prilocaine, articaine, ropivacaine, dibucaine and the mixtures thereof, and the said fatty acid is chosen from among the following: lauric acid or dodecanoic acid, tridecylic acid or tridecanoic acid, myristic acid or tetradecanoic acid, palmitic acid or hexadecanoic acid, stearic acid or octodecanoic acid, arachidic acid or eicosanoic acid, behenic acid or docosanoic acid, lignoceric acid or tetracosanoic acid, lauroleic acid or cis-9-dodecanoic acid, selacholeic acid or cis-15-tetracoseonic acid, g-linoleic acid or cis-cis-cis-6,9,12-octadecatrienoic acid and arachidonic acid or cis-cis-cis-cis-5,8,11,14-icosatetraenoic acid.

31. The oil phase (PHE) of claim 29 wherein the said local anaesthetic is lidocaine and the said fatty acid is selected from among lauric acid or tridecanoic acid.

32. The method of claim 1, wherein the pH of the hydrophilic phase is between 6.5 and 7.5.

33. The method of claim 9, wherein the said coil phase is in homogeneous liquid form and stable at a temperature of between 0° C. and 10° C.
